(19) United States
(12) Patent Application Publication
(10) Pub. No.: US 2015/0142079 A1
(43) Pub. Date: May 21, 2015

(54) Method for stimulating facial muscles

(71) Applicants: Jay Pensler, Chicago, IL (US); Christopherr Robinson, Elmwood Park, IL (US); Alexander Pensler, Chicago, IL (US)

(72) Inventors: Jay Pensler, Chicago, IL (US); Christopherr Robinson, Elmwood Park, IL (US); Alexander Pensler, Chicago, IL (US)

(21) Appl. No.: 14/085,532

(22) Filed: Nov. 20, 2013

(51) Int. Cl. A61N 1/36

(52) U.S. Cl. CPC A61N 1/36003
USPC 607/48

(57) ABSTRACT

A method for stimulating a facial muscle includes the steps of
mapping a facial nerve being connected to the facial muscle, locating the facial muscle in response to the mapping of the facial nerve and stimulating the facial nerve by applying an electrode and stimulating the facial nerve with current. The step of mapping includes the step of applying current to the cheek by an electrode and moving the electrode in response to the reaction of applying current to the cheek.

### FIELD OF THE INVENTION

The present invention relates to the stimulation of muscles and more particularly to the stimulation of facial muscles.

### BACKGROUND

Animals typically include muscles in order to achieve movement of the animal skeleton. Humans are no exception for using muscles in order to achieve motion, but muscles should be used in order to maintain the structural integrity of the muscles. Loss of structural integrity of the muscles is referred to as muscle atrophy. Muscle atrophy results from muscular inactivity for example muscle inactivity may occur as a result of the need for a cast to immobilize a portion of the skeleton. The muscle atrophy may be characteristically seen upon removal of a cast for bone stabilization after a fracture. Muscle atrophy also occurs with age. The muscle atrophy may be caused by Lipofuscin and fat being deposited in muscle with age or inactivity followed by replacement of the muscle with fibrous tissue. The result of the muscle atrophy is muscular weakness due reduced cell number and decreased mass of muscle with the loss of muscular tone.

All muscles contain two types of muscle fibers, type I fibers commonly referred to as slow twitch and type II fibers which are referred to as fast twitch. The most obvious changes with age and or inactivity are a decrease in the size of type II muscle fibers. In a normal muscle, the type II fibers are mostly larger than the type I fibers.

During a typical voluntary muscular contraction, the first muscle type to contract are type I fibers which are followed by the type II fibers with sustained and more forceful activity. During external electrical stimulation of the muscle, the order of recruitment for musculature is reversed i.e. the type II fibers initiate contraction followed by the type I fibers.

### SUMMARY

The facial nerve which controls the facial muscles exits the skull via the stylomastoid foramen which is behind the external auditory canal (the hole in the ear). The facial nerve courses or extends medially (towards the nose) under the ear and into the cheek. Once the facial nerve is positioned in the cheek, the facial nerve bifurcates into several branches: for example temporal, zygomatic, buccal, mandibular and cervical. The present invention places two electrodes on the face more particularly on the cheek. A first electrode is placed approximately 5 millimeters above and anterior to the external auditory canal, and the second electrode is placed approximately and initially 4.0 centimeters infermedial (towards the ear) towards the corner of the lip. The present invention then applies a series of Russian currents to the face via the two electrodes. While applying the currents, the reactions of the facial muscles are observed.

The present invention monitors the strength of contractions of the muscle groups at varying strengths of the electrical stimulation as the present invention moves the lower electrode.

The present invention following the use of Russian current employs biphasic electrical current to map out the branches of the facial nerve as it enters and stimulates the targeted facial muscles.

The stimulation of the facial muscles begins once the location of the facial muscles and appropriate branches of the facial nerve are identified. More particularly, the present invention stimulates the facial musculature in two ways. First, the present invention uses a Russian type of electrical current to directly stimulate and strengthen the facial musculature. Following this procedure, the present invention utilizes a Biphasic type of electrical current and a set of electrodes to locate the facial nerve and identify its branches and then directly stimulate the selected nerve branches to achieve contraction of the targeted facial muscles.

The present invention employs electrical current in sufficient strength to stimulate the facial muscles which has been previously detected to vigorously contract the facial muscles in order to eliminate the muscle atrophy. The present invention achieves the concentric contraction of the facial muscles with a set of electrodes. While the muscles of the face are contracting, the present invention then uses eccentric contraction to build and restore the volume of the muscles. Eccentric contraction as an example would be if you are performing an arm curl, the muscle activity on the way down (offload). In contrast, concentric contraction is the muscle activity on the way up for the weight (under load).

The activity builds up the resting tone of the facial muscles lifting the soft tissues of the face secondarily to the increased muscular tone and the increased muscular volume.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates a diagram of mapping steps of the present invention;

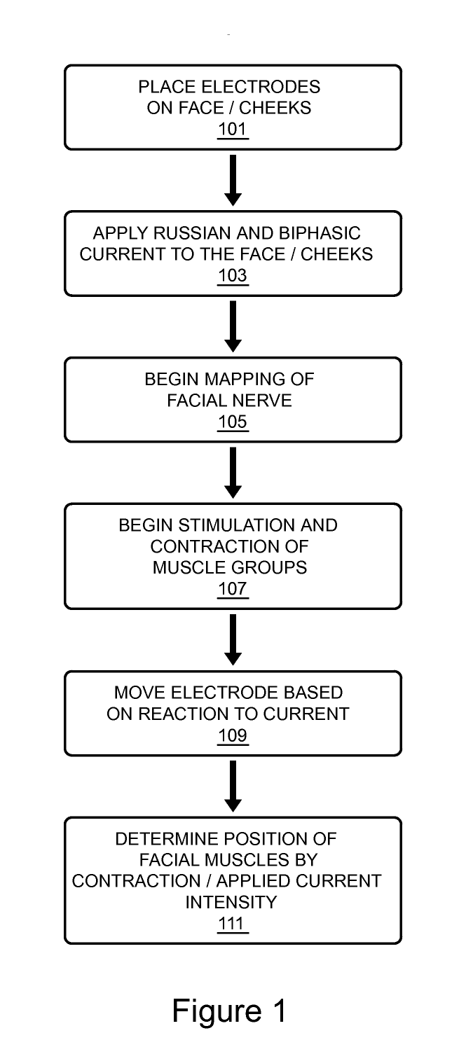

FIG. 2 illustrates a diagram of stimulation steps of the present invention.

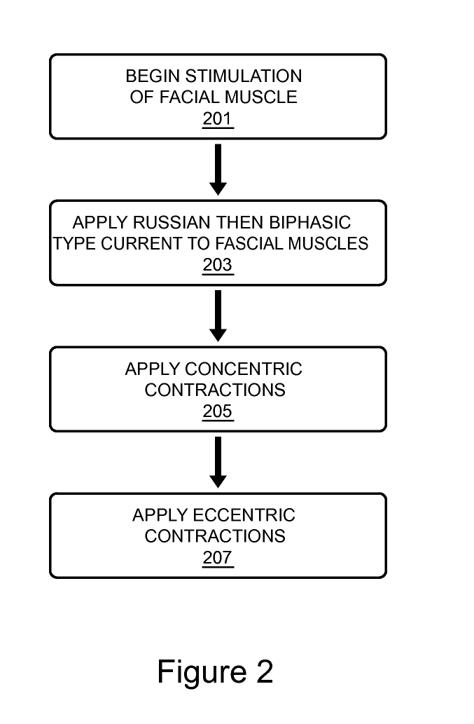

### DETAILED DESCRIPTION

The facial nerve, or cranial nerve (CN) VII, is the nerve responsible for the innervation of the muscles for facial expression.

The anatomy of the branches of the facial nerve in the cheek are variable, and knowledge of the location of the various branches is essential for a variety of interventions in the head and neck.

Muscle atrophy may be caused by Lipofuscin and fat being deposited in muscle with age or inactivity followed by replacement of the muscle with fibrous tissue. The result of the muscle atrophy results in muscular weakness due reduced cell number and decreased mass of muscle with the loss of muscular tone. The present invention will reverse these changes.

During the initial phase of treatment of the muscle atrophy, the present invention utilizes at least one of or both Russian and Biphasic current to stimulate the weakened facial musculature. The medial facial muscles may not be directly in the path of the Russian electrical stimulation. This initial treatment results in involuntary stimulation and strengthening of the lateral facial muscles referred to as the target muscles. The involuntary contraction is then coupled with volitional contraction of the target muscles.

The present process of the present invention may include first mapping of the facial nerve. 101 The facial nerve is employed to effectively stimulate the entire target muscles through the motor endplates of the nerve as opposed to direct stimulation of the muscle with the current. The indirect muscle stimulation via the facial nerve facilitates contraction of additional muscle groups 103 specifically the medial facial musculature.

The facial nerve which controls the facial muscles exits the skull via the style mastoid foramen which is behind the external auditory canal (the hole in the ear).

The facial nerve courses or extends medially (towards the nose) under the ear and into the cheek. Once the facial nerve is positioned in the cheek, the facial nerve bifurcates into several branches the temporal, zygomatic, buccal, mandibular and cervical. The present invention places two electrodes on the face more particularly on the cheek in step 105. A first electrode is placed approximately 5 millimeters above and anterior to the external auditory canal, and the second electrode is placed approximately and initially 1.5 centimeters lateral (towards the ear) to the corner of the lip. The present invention applies a series of biphasic currents to the face via the two electrodes in step 107. While applying the biphasic current, the reaction of the facial muscles is observed in order to determine the location of the facial muscles in step 109. Typically, the lower electrode is move closer to the ear, noting the contractions of the facial muscle groups based on the observed reaction of applying the current. The present invention monitors the strength of contractions of the muscle groups at varying strengths of the electrical stimulation as the present invention moves the lower electrode towards the ear. As the ratio of the strength of contractions/strength of electrical stimulation increases, the position of the facial muscles is approached in step 111. The present invention determines the near optimal position of the electrode (over the muscles) to provide maximal contractions of the targeted muscle groups via stimulation of the facial nerve at the near minimally required intensity of the electrical stimulation that is tolerable for the patients.

The present invention employs electrical current from both Russian and sinusoidal voltages to map out the facial nerve and muscles.

The mapping is complete.

The stimulation of the facial muscles begins in step 201. More particularly, the present invention stimulates the facial musculature in two ways. First, the present invention uses a Russian type of electrical current in step 203 to directly stimulate and strengthen the facial musculature. Following this procedure, the present invention utilizes a Biphasic type of electrical current and a set of electrodes to locate the facial nerve and identify its branches and then directly stimulate the selected nerve branches to achieve contraction of the targeted facial muscles. The present invention employs electrical current in sufficient strength to stimulate the facial muscles which has been previously detected to vigorously contract the facial muscles in order to eliminate the muscle atrophy. The present invention achieves the stimulation of the facial muscles with a set of electrodes. While the muscles of the face are contracting, the present invention uses eccentric contraction in step 205 to build and restore the muscles. Eccentric contraction as an example would be if you are performing an arm curl, the muscle activity on the way down (offload). In contrast, concentric contraction is the muscle activity on the way up for the weight (under load).

The activity builds up the resting tone of the facial muscles lifting the soft tissues of the face. The strength of the current may require a licensed physician MD to prescribe and perhaps to administer the procedure.

As the facial nerve branches are identified and the additional muscles stimulated the muscles are included in volitional contractions. The aforementioned activity results in the concentric contraction of the targeted musculature in the absence of resistance. The functional equivalent of the stimulation of the facial muscles at this stage in the weight room would be to perform an arm curl without a barbell.

To further facilitate muscular volume increase and strengthening along with increased resting tone of the targeted muscles, the present invention executes a series of eccentric muscle contractions in step 207. The targeted eccentric contractions reverse the recruitment pattern observed during myoelectric simulation, with the type I fibers initiating the eccentric activity followed closely by the large type II fibers. The functional equivalent of this activity in the weight room for the facial muscle would be to place a significant weight for example a fifty pound barbell in each arm with the wrist close to the biceps and then have individual slowly lower the weight as the elbow straightens.

The end result of the invention is a substantial positive change in volume, strength and resting tone of the targeted muscles. The visual repercussion of the enlarged muscular volume and resting tone is an observable fullness and elevation of the facial soft tissue matrix.

A series of Russian and Biphasic electrical currents are utilized via transcutaneous electrodes to map and stimulate the type I and type II muscle fibers of the facial musculature and the branches of the facial nerve. Once the location of the branches of the facial nerve and facial musculature are identified the musculature and nerve branches undergo serial stimulation with progressively higher amplitudes and frequencies of electrical current for increased durations of time. The stimulation is modulated to achieve an observed physiologic response of appropriate sustained concentric muscular contractions which are followed by group specific sustained eccentric muscular contractions in order to recruit both type I and type II fibers of the various facial muscles. The end result is an increase in the volume and resting tone of specific facial musculature and concomitant elevation of the overlying soft tissue.

The invention relates to:
1) Initial stimulation of the facial musculature with direct electrical current.
2) Mapping the Facial and Trigeminal nerves of the face to facilitate additional muscle stimulation and avoid pain during treatment.
3) Strengthening the identified facial type I and type II musculature via: 1) Progressive stimulation, 2) Concentric contraction and 3) Eccentric contraction.
4) The Facial musculature post treatment exhibits increased volume and resting tone.
5) The increased muscular volume will augment the soft tissue envelope of the face.
6) The facial musculature's increased resting tone will vertically reposition the muscular dermal attachments and the overlying skin.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1. A method for stimulating a facial muscle, comprising the steps of:
mapping a facial nerve being connected to the facial muscle;
locating the facial muscle in response to the mapping of the facial nerve;
stimulating the facial nerve by applying an electrode and stimulating the facial nerve with current;
wherein the step of mapping includes applying current to the cheek by an electrode and moving the electrode in response to the reaction of applying current to the cheek.

2. A method for stimulating a facial muscle as in claim 1, wherein the mapping step includes applying a biphasic current.

3. A method for stimulating a facial muscle as in claim 1, wherein the stimulation step includes applying a Russian current.

4. A method for stimulating a facial muscle as in claim 1, wherein the mapping step includes monitoring the strength of contractions.
